SEQUENCE LISTING (16 sequences)

SEQ ID NO: 1 - DNA, 396 bp, synthetic construct
  source 1..396: mol_type = other DNA
atgaagagctgcatttatacaaaattagttggcgagaggacttctctaggagcaactcgaagaaaacttccgcagaaacgaggaaataatgacaggaaattaagcaggagcgtcaaaacgataagagctgacatggttgagatcagcgagggacaaaaacgcataagagagggacagaaggaaatcaggaaaagatttcaagaaataagcgaagagaccgccaaactgagagaggaaactaacgtaatctccaagcagagctctgaaaatcaactcaggcttgatctaatgtttcaaatcgtcaaagcaagagcagagaatgattatgccaaagacgccttgctcactcaaaccttacggttagtcatcctccaaatctttgtgccttgtgtttaa

SEQ ID NO: 2 - protein, 131 aa, synthetic construct
MKSCIYTKLVGERTSLGATRRKLPQKRGNNDRKLSRSVKTIRADMVEISEGQKRIREGQKEIRKRFQEISEETAKLREETNVISKQSSENQLRLDLMFQIVKARAENDYAKDALLTQTLRLVILQIFVPCV

SEQ ID NO: 3 - DNA, 55 bp, synthetic construct
  source 1..55: mol_type = other DNA
atttggagagaacacgggggactttgcaacatgaagagctgcatttatacaaaat

SEQ ID NO: 4 - DNA, 55 bp, synthetic construct
  source 1..55: mol_type = other DNA
tttgtagtctccgtcgtggtctttgtaatcaacacaaggcacaaagatttggagg

SEQ ID NO: 5 - DNA, 45 bp, synthetic construct
  source 1..45: mol_type = other DNA
atttacaattaccatggggcgcgccaacgaggaaataatgacagg

SEQ ID NO: 6 - DNA, 48 bp, synthetic construct
  source 1..48: mol_type = other DNA
acataagaaattcttacacatttaaatgtgagcaaggcgtctttggca

SEQ ID NO: 7 - DNA, 45 bp, synthetic construct
  source 1..45: mol_type = other DNA
gtcaatttgcaggtatttggatccgtgagcaaggcgtctttggca

SEQ ID NO: 8 - DNA, 43 bp, synthetic construct
  source 1..43: mol_type = other DNA
cgggtcttaattaactctctagaaacgaggaaataatgacagg

SEQ ID NO: 9 - DNA, 22 bp, synthetic construct
  source 1..22: mol_type = other DNA
ctcctttgccccggagattaca

SEQ ID NO: 10 - DNA, 25 bp, synthetic construct
  source 1..25: mol_type = other DNA
ggggtaatgttgtttgttgtttgtt

SEQ ID NO: 11 - DNA, 20 bp, synthetic construct
  source 1..20: mol_type = other DNA
gagcatatacgcccggagtc

SEQ ID NO: 12 - DNA, 20 bp, synthetic construct
  source 1..20: mol_type = other DNA
caagacctgcctgaaaccga

SEQ ID NO: 13 - DNA, 20 bp, synthetic construct
  source 1..20: mol_type = other DNA
ccgcagaaacgaggaaataa

SEQ ID NO: 14 - DNA, 20 bp, synthetic construct
  source 1..20: mol_type = other DNA
ctcagtttggcggtctcttc

SEQ ID NO: 15 - DNA, 20 bp, synthetic construct
  source 1..20: mol_type = other DNA
cccattgagcacggtattgt

SEQ ID NO: 16 - DNA, 20 bp, synthetic construct
  source 1..20: mol_type = other DNA
tacgaccactggcatacagg

(19) United States
(12) Patent Application Publication
(10) Pub. No.: US 2026/0092286 A1
(43) Pub. Date: Apr. 2, 2026

(54) PagPB GENE REGULATING NITROGEN ABSORPTION AND UTILIZATION IN ROOT SYSTEM OF WOODY PLANT AND APPLICATION THEREOF

(71) Applicant: Research Institute of Forestry, Chinese Academy of Forestry, Beijing (CN)

(72) Inventors: Jing Zhou, Beijing (CN); Minglei Zhou, Beijing (CN); Dongliang Cao, Beijing (CN)

(21) Appl. No.: 19/283,275

(22) Filed: Jul. 29, 2025

(30) Foreign Application Priority Data
Sep. 27, 2024 (CN) 202411360505.9

(51) Int. Cl. C12N 15/82; C12N 15/66; C12N 15/74

(52) U.S. Cl. CPC C12N 15/8205; C12N 15/66; C12N 15/743; C12N 15/8227; C12N 15/8261

(57) ABSTRACT

A PagPB gene regulating nitrogen absorption and utilization in a root system of a woody plant is provided. The nucleotide sequence of the PagPB gene is shown in SEQ ID NO: 1. By silencing the PagPB gene, efficiency of the nitrogen absorption and utilization of a root system of poplar can be improved. In contrast, by overexpressing the PagPB gene, the efficiency of the nitrogen absorption and utilization of the root system of the poplar is reduced. This provides significant genetic resources for identification of key regulatory genes involved in the nitrogen absorption and utilization in the woody plant and holds important application value in the fields of forest genetic engineering and clonal forestry.

### CROSS-REFERENCE TO RELATED APPLICATION

This application claims priority to Chinese Patent Application No. 202411360505.9, filed on Sep. 27, 2024, which is herein incorporated by reference in its entirety.

### TECHNICAL FIELD

The disclosure relates to the field of genetic engineering breeding technologies, and more particularly to a PagPB gene regulating nitrogen absorption and utilization in a root system of a woody plant and an application thereof.

### STATEMENT REGARDING SEQUENCE LISTING

The sequence listing associated with this application is provided in text format in lieu of a paper copy and is hereby incorporated by reference into the specification. The name of the XML file containing the sequence listing is 25019MYZ-USP1-SL.xml. The XML file is 15,849 bytes; is created on Jul. 18, 2025; and is being submitted electronically via patent center.

### BACKGROUND

Nitrogen is one of the essential macronutrients required for plant growth and development. The efficiency of nitrogen absorption and utilization in a root system of a plant from soil is a key factor that constrains the plant growth and development. The root system of the plant needs to adapt to a complex and variable soil nitrogen environment, and the regulation of the efficiency of nitrogen absorption and utilization depends on changes in expression patterns of related genes, such as nitrate or ammonium transporter genes, protein-coding genes, and small ribonucleic acids (RNAs). Woody plants, as an important component of ecosystems, make significant contributions to ecosystem stability, agricultural production, biodiversity, carbon sequestration, stress resistance, molecular breeding, germplasm innovation, and sustainable agriculture and forestry. The woody plants maintain a soil nitrogen cycle through nitrogen fixation and nitrogen absorption, reduce fertilizer application, increase yield and quality of forest products, provide habitats for wildlife, enhance carbon sequestration capabilities, improve stress resistance, and cultivate superior varieties to achieve sustainable development in agriculture and forestry. Nowadays, most related research has been limited to herbaceous plants, and there is a lack of understanding regarding genes associated with nitrogen absorption and utilization in root systems of the woody plants.

### SUMMARY

In view of this, the disclosure provides a PagPB gene regulating nitrogen absorption and utilization in a root system of a wood plant and an application thereof, aiming to provide high-quality germplasm for breeding of new varieties of resource-efficient forest trees.

The disclosure is realized by the following technical solutions.

The disclosure provides a PagPB gene regulating nitrogen absorption and utilization in a root system of a woody plant, and the nucleotide sequence of the PagPB gene is shown in SEQ ID NO: 1.

The disclosure further provides an application of the PagPB gene in regulating the nitrogen absorption and utilization in the root system of the woody plant.

The application of the PagPB gene in regulating the nitrogen absorption and utilization in the root system of the woody plant is achieved by silencing/inhibiting the PagPB gene, thereby improving the efficiency of the nitrogen absorption and utilization in the root system of the woody plant and increasing biomass of the root system of the woody plant.

Improving the efficiency of the nitrogen absorption and utilization in the root system of the woody plant includes: increasing net nitrate ion (NO₃⁻) uptake rate and net ammonium ion (NH₄⁺) uptake rate in the root system of the woody plant, increasing nitrate content and ammonium content in the root system of the woody plant, and improving nitrate reductase (NR) activity in the root system of the woody plant.

The application of the PagPB gene in regulating the nitrogen absorption and utilization in the root system of the woody plant includes: constructing a silencing vector, and transforming the woody plant to obtain a PagPB gene-silenced transgenic plant, thereby improving the efficiency of the nitrogen absorption and utilization in the root system of the woody plant and increasing the biomass of the root system of the woody plant.

In an embodiment, a method for constructing the silencing vector of the PagPB gene includes:
(1) amplifying a full-length sequence of the PagPB gene by using PagPB gene sequence-specific primers to obtain a first polymerase chain reaction (PCR) product, and ligating the first PCR product into a T-vector to obtain a plasmid pM-PagPB;
(2) performing double-digestion on a pFGC5941 ribonucleic acid interference (RNAi) vector to obtain a double-digested pFGC5941 RNAi vector, amplifying the plasmid pM-PagPB by using a PagPB-cis-F primer and a PagPB-cis-R primer to obtain a second PCR product, and ligating the second PCR product with the double-digested pFGC5941 RNAi vector to obtain a ligation product; and introducing the ligation product into bacteria to obtain a recombinant vector pM-PagPB-C; and
(3) performing double-digestion on the recombinant vector pM-PagPB-C to obtain a double-digested recombinant vector pM-PagPB-C, amplifying the plasmid pM-PagPB by using a PagPB-anti-F primer and a PagPB-anti-R primer to obtain a third PCR product, and ligating the third PCR product with the double-digested recombinant vector pM-PagPB-C to obtain the silencing vector of the PagPB gene.

The sequences of the PagPB gene sequence-specific primers are shown in SEQ ID NO: 3 and SEQ ID NO: 4 respectively.

The sequence of the PagPB-cis-F primer is shown in SEQ ID NO: 5, and the sequence of the PagPB-cis-R primer is shown in SEQ ID NO: 6.

The sequence of the PagPB-anti-F primer is shown in SEQ ID NO: 7, and the sequence of the PagPB-anti-R primer is shown in SEQ ID NO: 8.

In an embodiment, a method of the transforming is an Agrobacterium-mediated method.

In an embodiment, the woody plant is a poplar plant.

In an embodiment, a variety of the poplar plant is poplar 84K (Populus alba x Populus glandulosa).

Compared to the related art, the disclosure may achieve the following beneficial effects.
(1) Discovery of a new gene: through screening and identification, the disclosure successfully discovers a new PagPB gene by using poplar 84K as material. This discovery enriches the genetic resources of poplar plants and provides a new direction for subsequent research.
(2) Revealing a new mechanism of the nitrogen absorption and utilization: the disclosure discovers that silencing the PagPB gene can improve the efficiency of the nitrogen absorption and utilization in the root system of the poplar plant, while overexpressing the PagPB gene reduces the efficiency of the nitrogen absorption and utilization in the root system of the poplar plant. The disclosure reveals a key role of the PagPB gene in regulating the nitrogen absorption and utilization in the root system of the poplar plant. This discovery provides a new strategy for in-depth analysis of molecular mechanisms of the nitrogen absorption and utilization in the woody plant.
(3) Providing high-quality germplasm: based on the nucleotide sequence of the PagPB gene, the disclosure design specific primers to construct the silencing vector of the PagPB gene. Through the mediation of Agrobacterium tumefaciens GV3101, the PagPB gene is transferred into the 84K poplar, and the PagPB gene-silenced transgenic plant is successfully generated, thereby providing the high-quality germplasm for the breeding of resource-efficient new poplar varieties in China.
(4) Significant application value: the disclosure has significant application value in the field of forestry genetic engineering, and can provide novel technological support for forest tree breeding, sustainable utilization of forest resources, and improvement of ecological environments.

### BRIEF DESCRIPTION OF DRAWINGS

To more clearly illustrate technical solutions described in embodiments of the disclosure or in the related art, the following is a brief description to attached drawings used in the description of the embodiments or the existing technologies. It is apparent that the attached drawings described below are merely some embodiments of the disclosure. For those skilled in the art, other attached drawings can be obtained based on these attached drawings without making inventive efforts.

FIG. 1 illustrates a schematic diagram of deoxyribonucleic acid (DNA) identification results of PagPB gene-silenced and PagPB gene-overexpressed transgenic lines according to the disclosure. Specifically, in FIG. 1, (a) illustrates that PagPB-RNAi1, 2, 3, 4, 16, 20, 21, and 22 are transgenic silencing lines; and (b) illustrates that PagPB-OE1 to PagPB-OE21 are transgenic overexpressing lines. Moreover, in FIG. 1, 84K represents a non-transgenic poplar negative control; and P represents a vector positive control.

FIG. 2A and FIG. 2B illustrate schematic diagrams of relative expression patterns of the PagPB gene in root systems of PagPB gene-silenced transgenic poplar plants and PagPB gene-overexpressed transgenic poplar plants, compared to wild-type poplar plants according to the disclosure. Specifically, FIG. 2A illustrates a schematic diagram of relative expression levels of the PagPB gene (also referred to as PB gene) in root systems of the PagPB gene-silenced transgenic poplar plants and the wild-type poplar plant. FIG. 2B illustrates a schematic diagram of relative expression levels of the PagPB gene in root systems of the PagPB gene-overexpressed transgenic poplar plants and the wild-type poplar plant.

FIGS. 3A-3D illustrate schematic diagrams of measurement results of net NO₃⁻ and NH₄⁺ uptake rates in the root systems of the PagPB gene-silenced transgenic poplar plants and the PagPB gene-overexpressed transgenic poplar plants, compared to the wild-type poplar plants according to the disclosure. Specifically, FIG. 3A illustrates a schematic diagram of measurement results of net NO₃ uptake rates in the root systems of the PagPB gene-silenced transgenic poplar plants and the wild-type poplar plant.

FIG. 3B illustrates a schematic diagram of measurement results of net NH₄⁺ uptake rates in the root systems of the PagPB gene-silenced transgenic poplar plants and the wild-type poplar plant. FIG. 3C illustrates a schematic diagram of measurement results of net NO₃⁻ uptake rates in the root systems of the PagPB gene-overexpressed transgenic poplar plants and the wild-type poplar plant. FIG. 3D illustrates a schematic diagram of measurement results of net NH₄⁺ uptake rates in the root systems of the PagPB gene-overexpressed transgenic poplar plants and the wild-type poplar plant.

FIG. 4 illustrates a schematic diagram of statistical results of biomass in the root systems of the PagPB gene-silenced transgenic poplar plants and the PagPB gene-overexpressed transgenic poplar plants, compared to the wild-type poplar plants according to the disclosure. Specifically, in FIG. 4, (a) illustrates statistical results of biomass in the root systems of the PagPB gene-silenced transgenic poplar plants and the wild-type poplar plant; and (b) illustrates statistical results of biomass in the root systems of the PagPB gene-overexpressed transgenic poplar plants and the wild-type poplar plant.

FIGS. 5A-5D illustrate schematic diagrams of statistical results of nitrate contents and ammonium contents in the root systems of the PagPB gene-silenced transgenic poplar plants and the PagPB gene-overexpressed transgenic poplar plants, compared to the wild-type poplar plants according to the disclosure. Specifically, FIG. 5A illustrates a schematic diagram of statistical results of nitrate contents in the root systems of the PagPB gene-silenced transgenic poplar plants and the wild-type poplar plant. FIG. 5B illustrates a schematic diagram of statistical results of ammonium contents in the root systems of the PagPB gene-silenced transgenic poplar plants and the wild-type poplar plant. FIG. 5C illustrates a schematic diagram of statistical results of nitrate contents in the root systems of the PagPB gene-overexpressed transgenic poplar plants and the wild-type poplar plant. FIG. 5D illustrates a schematic diagram of statistical results of ammonium contents in the root systems of the PagPB gene-overexpressed transgenic poplar plants and the wild-type poplar plant.

FIGS. 6A-6D illustrate schematic diagrams of measurement results of NR and glutamine synthetase (GS) activities in the root systems of the PagPB gene-silenced transgenic poplar plants and the PagPB gene-overexpressed transgenic poplar plants, compared to the wild-type poplar plants according to the disclosure. Specifically, FIG. 6A illustrates a schematic diagram of measurement results of NR activity in the root systems of the PagPB gene-silenced transgenic poplar plants and the wild-type poplar plant. FIG. 6B illustrates a schematic diagram of measurement results of GS activity in the root systems of the PagPB gene-silenced transgenic poplar plants and the wild-type poplar plant. FIG. 6C illustrates a schematic diagram of measurement results of NR activity in the root systems of the PagPB gene-overexpressed transgenic poplar plants and the wild-type poplar plant. FIG. 6D illustrates a schematic diagram of measurement results of GS activity in the root systems of the PagPB gene-overexpressed transgenic poplar plants and the wild-type poplar plant.

### DETAILED DESCRIPTION OF EMBODIMENTS

In order to facilitate the understanding of the disclosure, a comprehensive description is provided below, along with specific embodiments of the disclosure. However, the disclosure can be implemented in many different forms and is not limited to embodiments described herein. On the contrary, a purpose of providing these embodiments is to enable a more thorough and complete understanding of the disclosed content of the disclosure.

Unless otherwise defined, all technical and scientific terms used in this article have the same meanings as commonly understood by those skilled in the art to which the disclosure belongs. The terms used in the specification of the disclosure are for the purpose of describing the specific embodiments only and are not intended to limit the disclosure.

Terms “first”, “second” and “third” are only used for descriptive purposes and are not intended to indicate or imply relative importance or to indicate the quantity of technical features, but only to distinguish different PCR products.

Purchasing sources of reagents and instruments used in the disclosure are shown in Table 1.

TABLE 1

| Reagents and instruments | Purchasing sources |
|---|---|
| Restriction endonuclease Asc I | New England Biolabs, Massachusetts, |
| | USA, R0558S |
| Restriction endonuclease Swa I | New England Biolabs, Massachusetts, |
| | USA, R0604S |
| Restriction endonuclease BamH | Takara, Dalian, China, 1605 |
| I | |
| Restriction endonuclease Xba I | New England Biolabs, Massachusetts, |
| | USA, R0145V |
| Restriction endonuclease Bsa I | New England Biolabs, Massachusetts, |
| | USA, R3733V |
| Restriction endonuclease Eco | Shanghai Fertigene (Biotechnology Co., |
| 31I | Ltd.), China, ER0291 |
| Total RNA extraction kit | Beijing Genedite, China, R318-50 |
| Reverse transcription kit | Beijing Jumei, China, mf166-p-01 |
| TB Green quantitative reverse | Dalian TaKaRa, China, RR820A |
| transcription polymerase chain | |
| reaction (qRT-PCR) kit | |
| Non-invasive micro | AMERICAN MOON, NMT150-YG |
| measurement system | |
| Scanning ion-selective | Scanning ion-selective electrode |
| electrode technology | technology, SIET |
| NR activity testing kit | Suzhou KeMing Bio, China |
| GS testing kit | Suzhou KeMing Bio, China |

The disclosure uses poplar 84K as material and successfully discovers a new PagPB gene, thereby enriching genetic resources of poplar plants. The nucleotide sequence of the PagPB gene is shown in SEQ ID NO: 1, and the amino acid sequence encoded by the PagPB gene is shown in SEQ ID NO: 2.

SEQ ID NO: 1:
ATGAAGAGCTGCATTTATACAAAATTAGTTGGCGAGAGGACTTCTCTAG
GAGCAACTCGAAGAAAACTTCCGCAGAAACGAGGAAATAATGACAGGAA
ATTAAGCAGGAGCGTCAAAACGATAAGAGCTGACATGGTTGAGATCAGC
GAGGGACAAAAACGCATAAGAGAGGGACAGAAGGAAATCAGGAAAAGAT
TTCAAGAAATAAGCGAAGAGACCGCCAAACTGAGAGAGGAAACTAACGT
AATCTCCAAGCAGAGCTCTGAAAATCAACTCAGGCTTGATCTAATGTTT
CAAATCGTCAAAGCAAGAGCAGAGAATGATTATGCCAAAGACGCCTTGC
TCACTCAAACCTTACGGTTAGTCATCCTCCAAATCTTTGTGCCTTGTGT
TTAA.
SEQ ID NO: 2:
MKSCIYTKLVGERTSLGATRRKLPQKRGNNDRKLSRSVKTIRADMVEIS
EGQKRIREGQKEIRKRFQEISEETAKLREETNVISKQSSENQLRLDLMF
QIVKARAENDYAKDALLTQTLRLVILQIFVPCV.

Furthermore, the disclosure uses a homologous recombination method to construct a silencing vector and an overexpression vector of the PagPB gene respectively. The silencing vector and the overexpression vector of the PagPB gene are then introduced into the poplar 84K via an Agrobacterium-mediated method to generate PagPB-silenced and PagPB-overexpressed transgenic poplar plants respectively. A purpose of this is to investigate a regulatory role of the PagPB gene in nitrogen absorption and utilization of a poplar plant, aiming to provide high-quality new germplasm for the breeding of resource-efficient new poplar varieties in China.

In order to better illustrate the disclosure, the following embodiments will be used to further explain the content of the disclosure. Specific embodiments are as follows.

First embodiment: construction of the silencing vector and the overexpression vector of the PagPB gene
(1) PagPB gene sequence-specific primers are designed by using a Primer5 software, and a full-length sequence of the PagPB gene is amplified. The PagPB gene sequence-specific primers include a PagPB gene full-length amplification forward primer and a PagPB gene full-length amplification reverse primer. The sequence of the PagPB gene full-length amplification forward primer is shown in SEQ ID NO: 3, and the sequence of the PagPB gene full-length amplification reverse primer is shown in SEQ ID NO: 4.

SEQ ID NO: 3:
5′-atttggagagaacacgggggactttgcaacatgaagagctgcattt
atacaaaat-3′.
SEQ ID NO: 4:
5′-tttgtagtctccgtcgtggtctttgtaatcaacacaaggcacaaag
atttggagg-3′.

A high-fidelity PCR reaction system is as follows. Experimental materials include a high-fidelity amplification enzyme PrimeSTAR from TaKaRa, a forward primer, a reverse primer and a template derived from total RNA of a root system of 84K poplar. After full-length complementary deoxyribonucleic acid (cDNA) reverse transcription, full-length sequence amplification is carried out. A reaction program is: initial denaturation at 94° C. for 7 minutes; 94° C. for 30 seconds, 50° C. for 45 seconds, 72° C. for 24 seconds, for 30 cycles; 72° C. for 10 minutes, 16° C. for 30 minutes.

The full-length PagPB gene obtained is 396 base pairs (bp) in length, which is designated as the PagPB gene, shown in SEQ ID NO: 1.
(2) The 396 bp PagPB gene fragment is separated by 1.5% agarose gel electrophoresis at a voltage of 5 volts per centimeter (V/cm) for 20 minutes. An electrophoresis band corresponding to the 396 bp PagPB gene is excised under ultraviolet (UV) light, and a first PCR product is obtained. The first PCR product is then ligated into a T-vector to obtain a first sequencing-positive plasmid. The first sequencing-positive plasmid is designated as pM-PagPB.
(3) A pFGC5941 RNAi vector is subjected to double-digestion with restriction endonucleases Asc I and Swa I to obtain a double-digested pFGC5941 RNAi vector, and the double-digestion is reacted at 37° C. in a 10 microliters (μL) reaction volume for 1 hour. The plasmid pM-PagPB is amplified by using a PagPB-cis-F primer and a PagPB-cis-R primer to obtain a second PCR product. The second PCR product is ligated to the double-digested pFGC5941 RNAi vector to obtain a ligation product. The sequence of the PagPB-cis-F primer is shown in SEQ ID NO: 5, and the sequence of the PagPB-cis-R primer is shown in SEQ ID NO: 6. The ligation product is then introduced into Escherichia coli DH5a by using a heat-shock transformation method. Single colonies are selected for sequencing. A plasmid from a positively detected colony is retained to obtain a second sequencing-positive plasmid, and the second sequencing-positive plasmid is designated as pM-PagPB-C (also referred to as recombinant plasmid pM-PagPB-C).

The sequence of the PagPB-cis-F primer, as shown in SEQ ID NO: 5, is: 5′-atttacaattaccatggggcgcgccAACGAGGAAATAATGACAGG-3′.

The sequence of the PagPB-cis-R primer, as shown in SEQ ID NO: 6, is: 5′-acataagaaattcttacacatttaaatGTGAGCAAGGCGTCTTTGGCA-3′.
(4) The recombinant plasmid pM-PagPB-C is subjected to double-digestion with restriction endonucleases BamH I and Xba I to obtain a double-digested recombinant vector pM-PagPB-C, and the double-digestion is reacted at 37° C. in a 10 μL reaction volume for 1 hour. The plasmid pM-PagPB is amplified by using a PagPB-anti-F primer and a PagPB-anti-R primer to obtain a third PCR product. The third PCR product is ligated to the double-digested recombinant plasmid pM-PagPB-C to obtain the silencing vector of the PagPB gene. The sequence of the PagPB-anti-F primer is shown in SEQ ID NO: 7, and the sequence of the PagPB-anti-R primer is shown SEQ ID NO: 8. Then the recombinant plasmid (i.e., the silencing vector of the PagPB gene) is further introduced into Agrobacterium tumefaciens GV3010 by using a freeze-thaw method, thereby to obtain a PagPB-silenced recombinant Agrobacterium.

The sequence of the PagPB-anti-F primer, as shown in SEQ ID NO: 7, is: 5′-gtcaatttgcaggtatttggatccGTGAGCAAGGCGTCTTTGGCA-3′.

The sequence of the PagPB-anti-R primer, as shown in SEQ ID NO: 8, is: 5′-cgggtcttaattaactctctagaAACGAGGAAATAATGACAGG-3′.
(5) The full-length PagPB gene obtained in step (1) is separated by 1.5% agarose gel electrophoresis at a voltage of 5 V/cm for 20 minutes. An electrophoresis band corresponding to the 396 bp PagPB gene is excised under UV light, and a DNA fragment is recovered from the gel. The sequence accuracy of the DNA fragment is then verified.
(6) A pBWA(V)HS-ccdB-3×flag overexpression vector is subjected to digestion with restriction endonucleases Bsa I and Eco31I. The digestion is reacted in a 20 μL reaction volume at 37° C. for 1 hour, yielding a linearized pBWA(V)HS-ccdB-3×flag vector.
(7) The full-length sequence of the PagPB gene confirmed to be correct in step (5) is connected to the linearized pBWA(V)HS-ccdB-3×flag vector obtained in step (6) by using the homologous recombination method to obtain a ligation product.
(8) By using the heat-shock transformation method, the ligation product in step (7) is introduced into Escherichia coli DH5a competent cells, and then transformed onto a kanamycin-resistant plate. After incubation at 37° C. for 12 hours, colony PCR is performed for identification. Positive plasmids are then transformed into Agrobacterium tumefaciens GV3101, yielding a pBWA(V)HS-PB overexpressing recombinant Agrobacterium.

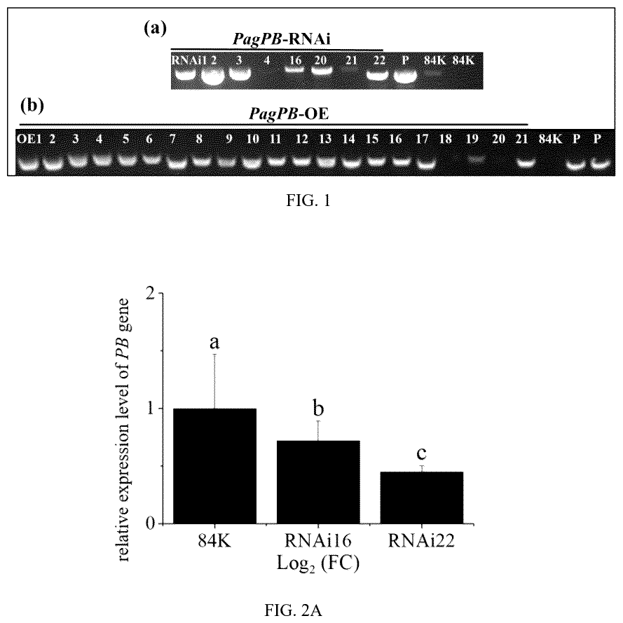

Second embodiment: creation of a PagPB gene-silenced transgenic poplar plant and a PagPB gene-overexpressed transgenic poplar plant
(1) 84K poplar tissue culture seedlings are cultured under conditions of 16 hours of daylight and a culture temperature of 25° C. The cultures containing the PagPB-silenced recombinant Agrobacterium and the pBWA(V)HS-PB overexpressing recombinant Agrobacterium are each cultured until an optical density at 600 nanometers (OD₆₀₀) reaches 0.6-0.8. Then, 10 micromoles per liter (μM) acetosyringone (AS) is added, and the cultures are further incubated on a shaker at 28° C. for 30 minutes before being set aside for subsequent use.
(2) Infection is carried out by using a leaf disk method. After infection, leaf disks are cultured in the dark for 4 days. Subsequently, the leaf disks are sterilized and transferred to selection media for PagPB gene-silenced poplar and PagPB gene-overexpressed poplar respectively. Under conditions of 16 hours of daylight and a culture temperature of 24° C., the induction and selection of a PagPB gene-silenced resistant adventitious bud and a PagPB gene-overexpressed resistant adventitious bud are conducted.
(3) A PagPB gene-silenced adventitious bud explant and a PagPB gene-overexpressing adventitious bud explant are respectively transferred to rooting media for the PagPB gene-silenced poplar plant and the PagPB gene-overexpressed poplar plant for further cultivation.
(4) DNA is extracted from leaves of PagPB gene-silenced transgenic poplar plants, PagPB gene-overexpressed transgenic poplar plants, and wild-type poplar plants by using a CTAB method. Using a leaf DNA as a template, PCR is performed with a pFGC5941-F primer (also referred to as pFGC5941 forward primer) with the sequence as shown in SEQ ID NO: 9 and a pFGC5941-R primer (also referred to as pFGC5941 reverse primer with the sequence as shown in SEQ ID NO: 10 to identify the PagPB gene-silenced transgenic plants. For the PagPB gene-overexpressed transgenic poplar plants, PCR is performed with a hygromycin resistance gene forward primer (with the sequence as shown in SEQ ID NO: 11) and a hygromycin resistance gene reverse primer (with the sequence as shown in SEQ ID NO: 12) carried on a vector to identify the PagPB gene-overexpressed transgenic plants. A positive control is a PagPB gene silencing recombinant plasmid vector or a PagPB gene overexpressing recombinant plasmid vector, and a negative control is the wild-type poplar plant. As shown in FIG. 1, electrophoresis results for transgenic silencing lines are Pag PBRNAi1, 2, 3, 4, 16, 20, 21, and 22, and electrophoresis results for the transgenic overexpressing line is OE1 to OE21. P represents the positive control of the PagPB gene silencing recombinant plasmid vector or the PagPB gene overexpressing recombinant plasmid vector, and 84K represents a negative control of the wild-type poplar plant.

The sequence of the pFGC5941 forward primer is shown in SEQ ID NO: 9 in the sequence list, as presented in Table 2 below.

TABLE 2

| Name | SEQ ID NO: 9 |
|---|---|
| pFGC5941forward | 5′-CTCCTTTGCCCCGGAGATTACA-3′ |
| primer | |

The sequence of the pFGC5941 reverse primer is shown in SEQ ID NO: 10 in the sequence list, as presented in Table 3 below.

TABLE 3

| Name | SEQ ID NO: 10 |
|---|---|
| pFGC5941 reverse | 5′-GGGGTAATGTTGTTTGTTGTTTGTT-3′ |
| primer | |

The sequence of the forward primer for the hygromycin resistance gene is shown in SEQ ID NO: 11 in the sequence list, as presented in Table 4 below.

TABLE 4

| Name | SEQ ID NO: 11 |
|---|---|
| Hygromycin resistance | 5′-gagcatatacgcccggagtc-3′ |
| gene forward primer | |

The sequence of the reverse primer for the hygromycin resistance gene is shown in SEQ ID NO: 12 in the sequence list, as presented in Table 5 below.

TABLE 5

| Name | SEQ ID NO: 12 |
|---|---|
| Hygromycin resistance | 5′-caagacctgcctgaaaccga-3′ |
| gene reverse primer | |

(5) Using root system samples of the gene-silenced transgenic poplar plants and gene-overexpressed transgenic poplar plants, as well as the wild-type poplar plant, total RNAs are extracted from the above samples by using a total RNA extraction kit and then reverse transcribed by using a reverse transcription kit. Subsequently, RT-qPCR is performed by using a TB Green qRT-PCR kit.

The sequence of a forward primer for quantitative analysis of the PagPB gene is shown in SEQ ID NO: 13 in the sequence listing, as presented in Table 6 below.

TABLE 6

| Name | SEQ ID NO: 13 |
|---|---|
| PagPB gene quantitative | 5′-CCGCAGAAACGAGGAAAT |
| forward primer | AA-3′ |

The sequence of a reverse primer for quantitative analysis of the PagPB gene is shown in SEQ ID NO: 14 in the sequence listing, as presented in Table 7 below.

TABLE 7

| Name | SEQ ID NO: 14 |
|---|---|
| PagPB gene quantitative | 5′-CTCAGTTTGGCGGTCTCT |
| reverse primer | TC-3′ |

Actin is used as a reference gene, the sequence of a forward primer for quantitative analysis of Actin is shown in SEQ ID NO: 15 in the sequence listing, as presented in Table 8 below.

TABLE 8

| Name | SEQ ID NO: 15 |
|---|---|
| Actin gene quantitative | 5′-CCCATTGAGCACGGTATT |
| forward primer | GT-3′ |

The sequence of a reverse primer for quantitative analysis of Actin is shown in SEQ ID NO: 16 in the sequence listing, as presented in Table 9 below.

TABLE 9

| Name | SEQ ID NO: 16 |
|---|---|
| Actin gene quantitative | 5′-TACGACCACTGGCATACAG |
| reverse primer | G-3′ |

The PCR program is as follows: 98° C. for 10 seconds; 94° C. for 15 seconds, 60° C. for 30 seconds, for 40 cycles. An expression level of the PagPB gene is calculated using a 2^{-ΔΔ/Ct} method. Statistical analysis is performed by using a t-test. A log₂(FC)≥1 or ≤−1 and P<0.05 are defined as statistically significant. The expression patterns of the PagPB gene in transgenic and wild-type plants are analyzed.

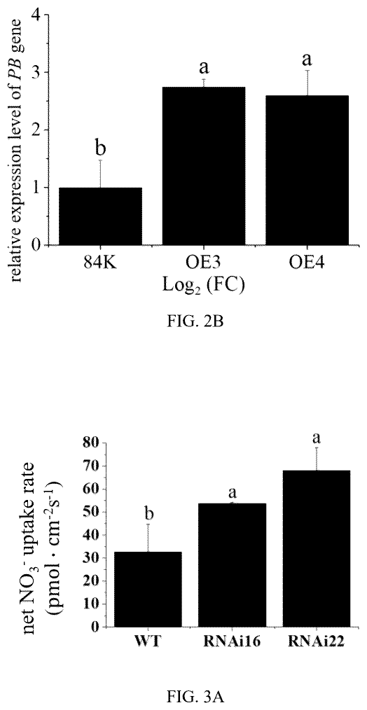

As shown in FIG. 2, relative expression levels of the PagPB gene in root systems of PagPB gene-silenced transgenic poplar lines (RNAi16 and RNAi22), PagPB gene-overexpressed transgenic poplar lines (OE3 and OE4), and wild-type 84K poplar are compared. Compared to the wild-type 84K poplar, the expression levels of the PagPB gene in the PagPB gene-silenced transgenic poplar lines (RNAi16 and RNAi22) are significantly lower than those of the wild-type control, while the expression levels of the PagPB gene in the root systems of the PagPB gene-overexpressed transgenic poplar lines (OE3 and OE4) are significantly increased.

Third embodiment: functional analysis of the PagPB gene in regulating the nitrogen absorption and utilization in the root system of the poplar

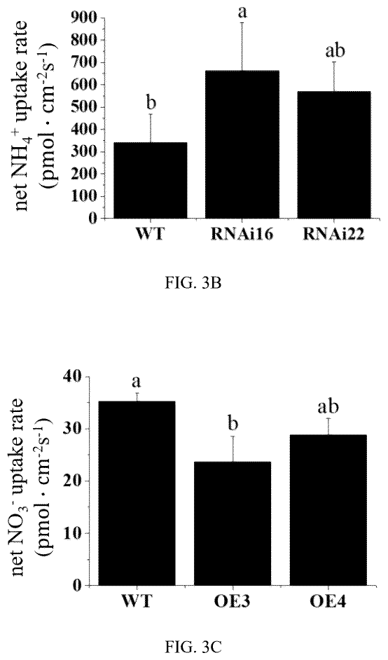

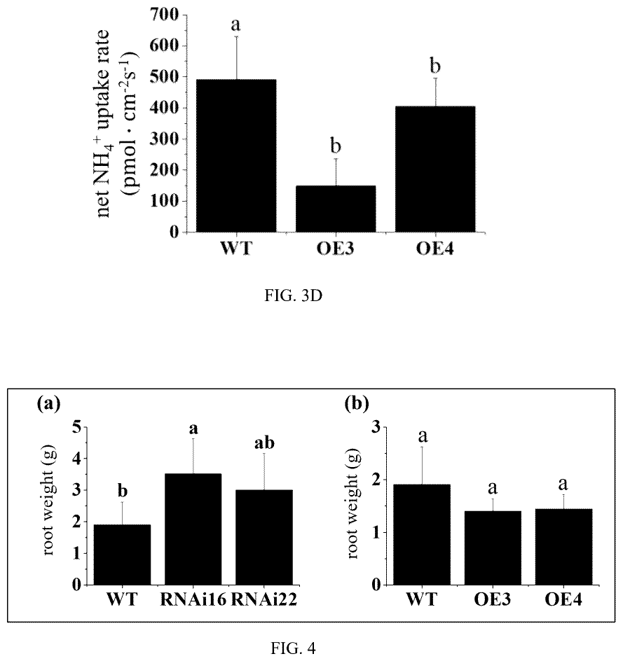

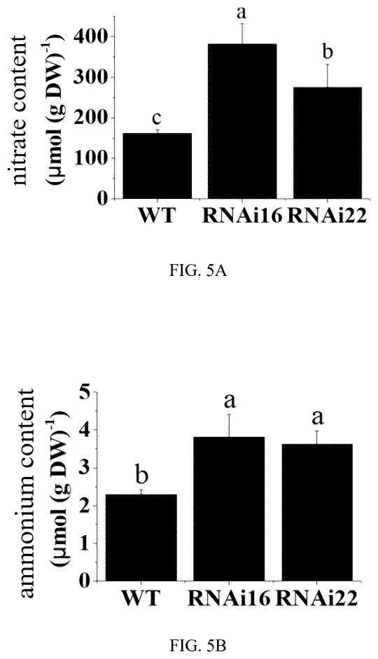

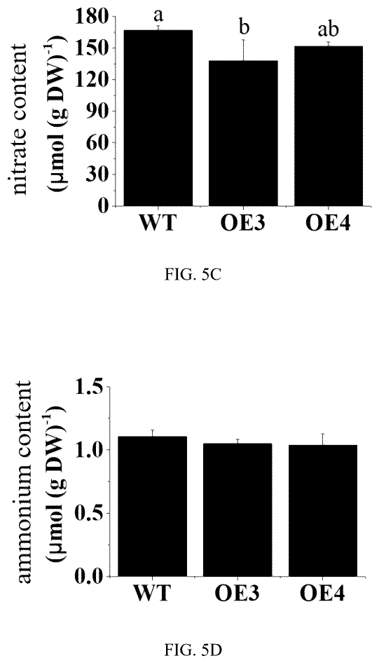

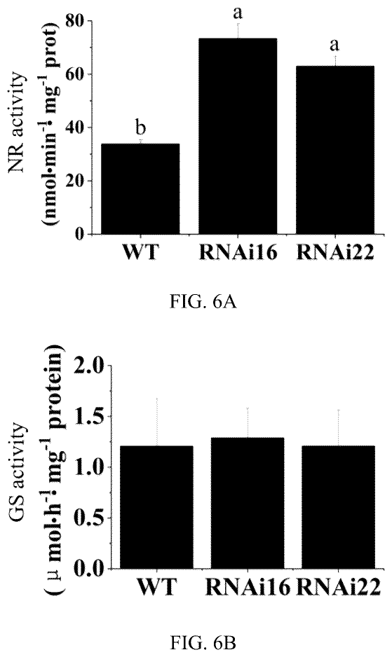

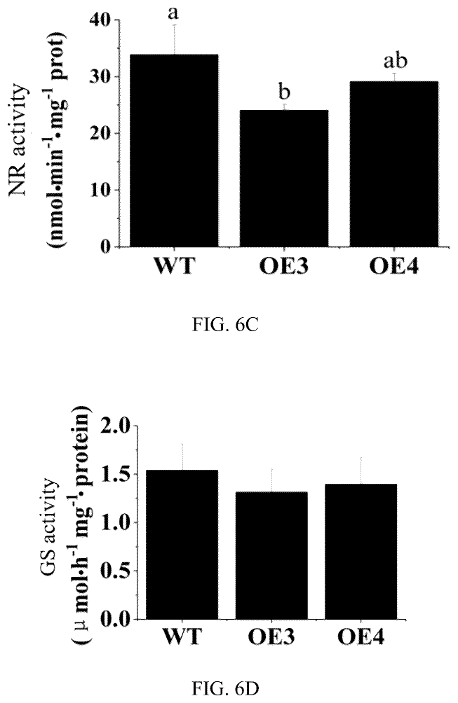

In this embodiment, net NO₃⁻ uptake rate and net NH₄⁺ uptake rate in the root system, biomass of the root system, nitrate content and ammonium content in the root system, as well as NR activity and GS activity in the root system are analyzed for the PagPB gene-silenced transgenic lines, PagPB gene-overexpressed transgenic lines, and wild-type plants. Steps are as follows.
(1) Transgenic poplar plants are obtained according to the method of the second embodiment. Using 3-month-old hydroponically grown seedlings of PagPB gene-silenced transgenic lines, PagPB gene-overexpressed transgenic lines and wild-type lines as experimental materials, net NO₃⁻ and NH₄⁺ uptake rates at a position 10 mm away from a root tip are determined by a non-invasive micro measurement system based on scanning ion-selective electrode technology, and results are shown in FIG. 3. Compared to the wild-type poplar 84K, the net NO₃⁻ uptake rates in the root systems of the PagPB gene-silenced transgenic lines RNAi16 and RNAi22 are significantly increased, and the net NH₄⁺ uptake rates are also increased. In contrast, the net NO₃⁻ uptake rates in the root systems of the PagPB gene-overexpressed transgenic lines OE3 and OE4 are decreased, and the net NH₄⁺ uptake rates are significantly decreased.
(2) Root system samples from the PagPB gene-silenced transgenic lines, PagPB gene-overexpressed transgenic lines, and the wild-type line are harvested, and the biomass of the root system of each of the transgenic and wild-type plants is measured, and results are shown in FIG. 4. Compared to the wild-type poplar 84K, the biomass of the root system of each of the PagPB gene-silenced transgenic poplar lines RNAi16 and RNAi22 is increased, while the biomass of the root system of each of the PagPB gene-overexpressed transgenic poplar lines OE3 and OE4 is decreased.
(3) The root system samples from the PagPB gene-silenced transgenic lines, PagPB gene-overexpressed transgenic lines, and the wild-type line are harvested to determine the nitrate contents and the ammonium contents, and results are shown in FIG. 5. Compared to the wild-type poplar 84K, the nitrate contents and the ammonium contents in the root systems of the PagPB gene-silenced transgenic poplar lines RNAi16 and RNAi22 are significantly increased, while the nitrate contents and the ammonium contents in the root systems of the PagPB gene-overexpressed transgenic poplar lines OE3 and OE4 are decreased.
(4) The root system samples are from the PagPB gene-silenced transgenic lines and PagPB gene-overexpressed transgenic lines, and the wild-type line are harvested. NR and GS activities are measured using a NR activity testing kit and a GS testing kit, and results are shown in FIG. 6. Compared to the wild-type poplar 84K, the NR activity in the root systems of the PagPB gene-silenced transgenic poplar lines RNAi16 and RNAi22 are significantly increased. In contrast, the NR activity in the root systems of the PagPB gene-overexpressed transgenic poplar lines OE3 and OE4 are decreased.

In conclusion, the disclosure involves the introduction of the PagPB gene into the 84K poplar. Compared to the wild-type poplar, PagPB gene-silenced transgenic poplar exhibits increased net NO₃⁻ and NH₄⁺ uptake rates, increased biomass of the root system, and significantly increased nitrate content and ammonium content, and enhanced nitrate reductase activity. In contrast, the PagPB gene-overexpressed transgenic poplar shows decreased net NO₃⁻ and NH₄⁺ uptake rates, significantly reduced nitrate content in the root system, and decreased ammonium content and nitrate reductase activity. These results indicate that silencing the PagPB gene can improve the efficiency of the nitrogen absorption and utilization of the root system of the poplar, while overexpressing the PagPB gene reduces the efficiency of the nitrogen absorption and utilization of the root system of the poplar. This demonstrates that the PagPB gene is a key regulatory gene for the nitrogen absorption and utilization in the root system of the poplar, particularly for nitrate. The disclosure has significant application value in the field of forest genetic engineering and clonal forestry.

Technical features described in the above embodiments can be combined in any manner. In order to make the description concise, not all possible combinations of the technical features in the above embodiments have been described. However, any combination of these technical features that does not result in a contradiction should be considered as within the scope of the disclosure.

The embodiments described above merely represent several implementations of the disclosure, and are described in a relatively specific and detailed manner. However, this should not be construed as limiting the scope of the disclosure. It should be pointed out that for those skilled in the art, various modifications and improvements can be made without departing from the concept of the disclosure, and these all fall within the scope of protection of the disclosure. Therefore, the scope of protection of the disclosure should be determined by the appended claims.

What is claimed is:

1. A PagPB gene regulating nitrogen absorption and utilization in a root system of a woody plant, wherein the nucleotide sequence of the PagPB gene is shown in SEQ ID NO: 1.

2. An application of the PagPB gene as claimed in claim 1 in regulating nitrogen absorption and utilization in a root system of poplar 84K (Populus alba x Populus glandulosa) comprising: silencing the PagPB gene, thereby improving efficiency of the nitrogen absorption and utilization in the root system of the poplar 84K and increasing biomass of the root system of the poplar 84K.

3. The application of the PagPB gene in regulating the nitrogen absorption and utilization in the root system of the poplar 84K as claimed in claim 2, wherein improving the efficiency of the nitrogen absorption and utilization in the root system of the poplar 84K comprises: increasing a net nitrate ion (NO₃⁻) rate and a net ammonium ion (NH₄⁺) uptake rate in the root system of the poplar 84K, increasing a nitrate content and an ammonium content in the root system of the poplar 84K, and improving nitrate reductase (NR) activity in the root system of the poplar 84K.

4. The application of the PagPB gene in regulating the nitrogen absorption and utilization in the root system of the poplar 84K as claimed in claim 2, comprising: constructing a silencing vector of the PagPB gene, and transforming the poplar 84K using the silencing vector of the PagPB gene to obtain a PagPB gene-silenced transgenic plant, thereby improving the efficiency of the nitrogen absorption and utilization in the root system of the poplar 84K and increasing the biomass of the root system of the poplar 84K.

5. The application of the PagPB gene in regulating the nitrogen absorption and utilization in the root system of the poplar 84K as claimed in claim 4, wherein a method for constructing the silencing vector of the PagPB gene comprises:
(1) amplifying a full-length sequence of the PagPB gene by using PagPB gene sequence-specific primers to obtain a first polymerase chain reaction (PCR) product, and ligating the first PCR product into a T-vector to obtain a plasmid pM-PagPB;
(2) performing double-digestion on a pFGC5941 ribonucleic acid interference (RNAi) vector to obtain a double-digested pFGC5941 RNAi vector, amplifying the plasmid pM-PagPB by using a PagPB-cis-F primer and a PagPB-cis-R primer to obtain a second PCR product, and ligating the second PCR product to the double-digested pFGC5941 RNAi vector to obtain a ligation product; and introducing the ligation product into bacteria to obtain a recombinant vector pM-PagPB-C; and
(3) performing double-digestion on the recombinant vector pM-PagPB-C to obtain a double-digested recombinant vector pM-PagPB-C, amplifying the plasmid pM-PagPB by using a PagPB-anti-F primer and a PagPB-anti-R primer to obtain a third PCR product, and ligating the third PCR product with the double-digested recombinant vector pM-PagPB-C to obtain the silencing vector of the PagPB gene;
wherein the sequences of the PagPB gene sequence-specific primers are shown in SEQ ID NO: 3 and SEQ ID NO: 4 respectively;
wherein the sequence of the PagPB-cis-F primer is shown in SEQ ID NO: 5, and the sequence of the PagPB-cis-R primer is shown in SEQ ID NO: 6; and
wherein the sequence of the PagPB-anti-F primer is shown in SEQ ID NO: 7, and the sequence of the PagPB-anti-R primer is shown in SEQ ID NO: 8.

6. The application of the PagPB gene in regulating absorption and utilization of nitrogen in the root system of the poplar 84K as claimed in claim 4, wherein a method of the transforming is an Agrobacterium-mediated method.
